(19) United States
(12) Patent Application Publication
(10) Pub. No.: US 2026/0283484 A1
(43) Pub. Date: Sep. 24, 2026

(54) Method and Apparatus with Multi-Wavelength Optical Instruments for Blood Pressure Measurement

(71) Applicant: University of Virginia Patent Foundation, Charlottesville, VA (US)

(72) Inventors: Amanda Watson, Charlottesville, VA (US); Tarek Hamid, Freehold, NJ (US)

(21) Appl. No.: 19/575,442

(22) Filed: Mar. 23, 2026

Related U.S. Application Data
(60) Provisional application No. 63/775,703, filed on Mar. 21, 2025.

(51) Int. Cl. A61B 5/021; A61B 5/00

(52) U.S. Cl. CPC A61B 5/02108; A61B 5/6824; A61B 5/6826; A61B 5/7267

(57) ABSTRACT

An apparatus for measuring blood pressure can be positioned on a subject in a position that is proximate to blood vessels. A plurality of LEDs direct a plurality of wavelengths of light from the apparatus toward the identified blood vessels. A plurality of photodetectors generate a plurality of respective photoplethysmography (PPG) waveforms in response to each of the wavelengths of light. Software implemented by a computer develop a feature vector of wavelength data and PPG data and extracts feature values from the feature vector. The software calculates feature ratios of selected pairs of feature values that each include a corresponding PPG data value for a respective wavelength data value. Using the feature ratios as inputs in a machine learning model predicts systolic blood pressure (SBP), diastolic blood pressure (DBP), or mean arterial blood pressure (MAP) with the feature ratios.

### CROSS-REFERENCE TO RELATED APPLICATION

This application claims priority to and incorporates by reference U.S. Provisional Patent Application Ser. No. 63/775,703 filed on Mar. 21, 2025, and entitled System and Method for Multi-Wavelength Optical Sensing Framework for Calibration-Free Wearable Blood Pressure Monitoring.

### STATEMENT REGARDING FEDERALLY FUNDED RESEARCH

None.

### FIELD

This disclosure includes a blood pressure monitor for use with a subject as a wearable device and includes specialized machine learning software operating on feature data derived from optical signals sensed while monitoring blood vessels of the subject.

### BACKGROUND

Blood pressure is one of the most commonly monitored vital signs, critical for monitoring overall health [1]. Hypertension, or high blood pressure, is a leading contributor to morbidity and mortality in the United States, where nearly half of adults are affected [2]. The CDC has reported a stark increase in hypertension over the last decade, with prevalence rising from 10.2% in 2011-2012 to 48.1% in 2022[2 ]. Despite increased prevalence and awareness, blood pressure control remains unsatisfactory [3]. As a result, ubiquitous tools for detecting and managing blood pressure, especially outside the clinic, have become increasingly desired.

An effective approach to managing and reducing blood pressure-related diseases is early detection, timely treatment, and continuous monitoring. The oscillo-metric method, commonly used in clinical settings, relies on an inflatable cuff to measure oscillations in arterial wall pressure during deflation, similar to manual Korotkoff sound-based measurements [4]. However, this method has limitations, including discomfort from cuff inflation [5], inaccuracies in certain populations [6], improper usage [7], [8], and the inability to provide continuous monitoring [9]. These drawbacks have driven the development of cuffless methods to alleviate this burden.

To date, there has been a large amount of research on cuffless blood pressure monitoring; exhaustive reviews can be found in [10] and [11]. Traditionally, PPG is used alongside ECG to calculate pulse transit time (PTT), the time it takes for an arterial pulse wave to travel between two points. This measure correlates directly with blood pressure—shorter transit times generally indicate higher pressure due to stiffer arterial walls [12], [13]. However, systems based on PTT require initial and ongoing calibration with an oscillo-metric cuff to account for individual physiological differences, such as tissue composition and vascular elasticity [14], [15]. While this approach has enabled more continuous monitoring of blood pressure without the constant need for a cuff, it also faces challenges, including the ongoing need for cuff calibration [16], difficulty in calculating PTT with abnormal ECG signals [17], and the need for advanced algorithms to filter noise, especially in motion-sensitive PPG waveforms [18].

Recent research has increasingly focused on estimating blood pressure using advanced signal processing and machine learning techniques solely from PPG waveforms. These approaches extract features from fiducial points of the PPG waveform and utilize physiological models [19], machine learning regression [20], [21], and deep learning [21]-[23] to predict blood pressure with reasonable accuracy. Several calibration-free devices, such as those by Aktiia [24], Corsano [25], and Valencell [26], claim to provide accurate blood pressure readings using PPG and machine learning algorithms without the need for traditional cuff calibration. However, none of these devices has received FDA approval.

Despite promising progress in calibration-free PPG-based monitoring, challenges remain. Accurate predictions depend on the precise extraction of PPG waveform features, which requires high-fidelity data, a high sampling rate, and advanced denoising techniques—factors that can impact battery life in practical applications and may be impractical in a wearability constrained and noisy environment [27]. Additionally, deep learning models used in this context often lack explainability, a critical factor for clinical use.

A need still exists in the art of blood pressure monitoring for a convenient apparatus that may be attached to the patient easily and comfortably and take advantage of advances in machine learning algorithms.

### SUMMARY

Other aspects and features according to the example embodiments of the disclosed technology will become apparent to those of ordinary skill in the art, upon reviewing the following detailed description in conjunction with the accompanying figures.

In one implementation, a computer implemented method of measuring blood pressure (BP) of a subject includes placing a device on a surface of the subject in a position that is proximate to blood vessels identified for blood pressure measurements. The device directs a plurality of wavelengths of light from the device toward the identified blood vessels. The method continues by generating a plurality of respective photoplethysmography (PPG) waveforms and corresponding PPG data with the device in response to each of the wavelengths of light, and for each of the wavelengths of light, transmitting wavelength data and the corresponding PPG data from the device to a computer comprising a processor in communication with computer memory storing the wavelength data and the PPG data in a feature vector. The method further includes the processor conducting steps of extracting feature values from the feature vector; calculating feature ratios of selected pairs of feature values that each comprise a corresponding PPG data value for a respective wavelength data value; and using the feature ratios as inputs in a machine learning model to predict systolic blood pressure (SBP), diastolic blood pressure (DBP), or mean arterial blood pressure (MAP) with the feature ratios.

In another implementation, an apparatus for measuring blood pressure of a subject includes a form factor configured to place the apparatus on a surface of the subject in a position that is proximate to blood vessels identified for blood pressure measurements. A plurality of LEDs direct a plurality of wavelengths of light from the apparatus toward the identified blood vessels. A plurality of photodetectors generating a plurality of respective photoplethysmography (PPG) waveforms and corresponding PPG data in response to each of the wavelengths of light, and the photodetectors are configured to transmit, for each of the wavelengths of light, wavelength data and the corresponding PPG data from the apparatus to a computer comprising a processor in communication with computer memory storing the wavelength data and the PPG data in a feature vector. Software implemented by the computer perform steps including extracting feature values from the feature vector; calculating feature ratios of selected pairs of feature values that each comprise a corresponding PPG data value for a respective wavelength data value; and using the feature ratios as inputs in a machine learning model to predict systolic blood pressure (SBP), diastolic blood pressure (DBP), or mean arterial blood pressure (MAP) with the feature ratios.

### BRIEF DESCRIPTION OF THE DRAWINGS

Reference will now be made to the accompanying drawings, which are not necessarily drawn to scale.

FIG. 1 illustrates a blood pressure prediction framework according to aspects of this disclosure.

FIG. 2 is a schematic of a computer environment used in accordance with the disclosure herein.

FIG. 3 is a schematic of a computer environment used in accordance with the disclosure herein.

FIG. 4A is an apparatus disclosed according to this disclosure with a wristwatch form factor for wearing by the subject.

FIG. 4B is an apparatus disclosed according to this disclosure with a fingertip cuff form factor for wearing by the subject.

FIG. 5 is one example embodiment of an optical measurement system used according to aspects of this disclosure.

FIG. 6A illustrates a data distribution for systolic blood pressure measurements according to this disclosure.

FIG. 6B illustrates a data distribution for diastolic blood pressure measurements according to this disclosure.

FIG. 6C illustrates a data distribution for mean arterial blood pressure (MAP) according to this disclosure.

FIG. 7A is a portion of Table 1 showing subject-specific comparisons of various machine learning algorithms tested for accuracy in both finger and wrist form factors.

FIG. 7B is a portion of Table 1 showing generalized comparisons of various machine learning algorithms tested for accuracy in both finger and wrist form factors.

FIG. 8 is an illustration of Table 2 discussed herein as showing compliance with British Hypertension Society (BHS) compliance and grades for wrist and finger blood pressure measurements according to this disclosure.

FIG. 9 is an illustration of Table 3 discussed herein as showing compliance with IEEE and AAMI compliance and grades for wrist and finger blood pressure measurements according to this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. All references cited and discussed in this specification are incorporated herein by reference in their entireties and to the same extent as if each reference was individually incorporated by reference.

In the following description, references are made to the accompanying drawings that form a part hereof and that show, by way of illustration, specific embodiments or examples. In referring to the drawings, like numerals represent like elements throughout the several figures.

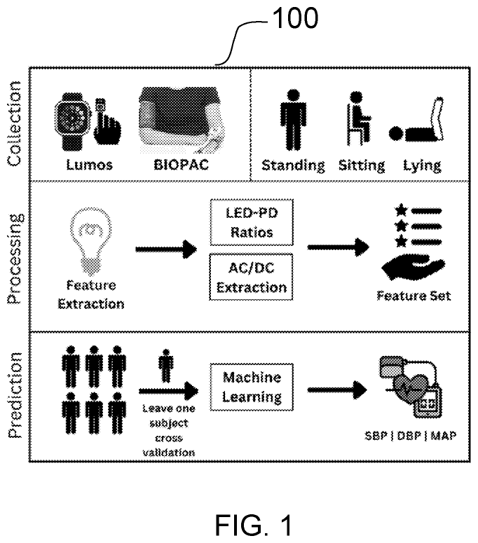

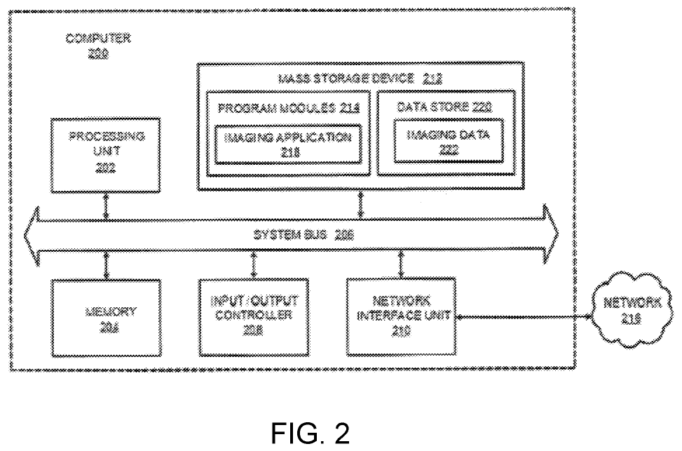

FIG. 2 is a computer architecture diagram showing a general computing system capable of implementing aspects of the present disclosure in accordance with one or more embodiments described herein. A computer 200 may be configured to perform one or more functions associated with embodiments of this disclosure. For example, the computer 200 may be configured to perform operations of the method as described below. It should be appreciated that the computer 200 may be implemented within a single computing device or a computing system formed with multiple connected computing devices. The computer 200 may be configured to perform various distributed computing tasks, which may distribute processing and/or storage resources among the multiple devices. The data acquisition and display computer 150 and/or operator console 110 of the system shown in FIG. 1 may include one or more systems and components of the computer 200.

As shown, the computer 200 includes a processing unit 202 (“CPU”), a system memory 204, and a system bus 206 that couples the memory 204 to the CPU 202. The computer 200 further includes a mass storage device 212 for storing program modules 214. The program modules 214 may be operable to perform one or more functions associated with embodiments of method as illustrated in one or more of the figures of this disclosure, for example to cause the computer 200 to perform operations of the automated DENSE analysis as described below. The program modules 214 may include an imaging application 218 for performing data acquisition functions as described herein, for example to receive image data corresponding to magnetic resonance imaging of an area of interest. The computer 200 can include a data store 220 for storing data that may include imaging-related data 222 such as acquired image data, and a modeling data store 224 for storing image modeling data, or other various types of data utilized in practicing aspects of the present disclosure.

The mass storage device 212 is connected to the CPU 202 through a mass storage controller (not shown) connected to the bus 206. The mass storage device 212 and its associated computer-storage media provide non-volatile storage for the computer 200. Although the description of computer-storage media contained herein refers to a mass storage device, such as a hard disk or CD-ROM drive, it should be appreciated by those skilled in the art that computer-storage media can be any available computer storage media that can be accessed by the computer 200.

By way of example, and not limitation, computer-storage media (also referred to herein as a “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 200. Transitory signals are not “computer-storage media”, “computer-readable storage medium” or “computer-readable storage media” as described herein.

According to various embodiments, the computer 200 may operate in a networked environment using connections to other local or remote computers through a network 216 via a network interface unit 210 connected to the bus 206. The network interface unit 210 may facilitate connection of the computing device inputs and outputs to one or more suitable networks and/or connections such as a local area network (LAN), a wide area network (WAN), the Internet, a cellular network, a radio frequency network, a Bluetooth-enabled network, a Wi-Fi enabled network, a satellite-based network, or other wired and/or wireless networks for communication with external devices and/or systems. The computer 200 may also include an input/output controller 208 for receiving and processing input from a number of input devices. Input devices may include one or more of keyboards, mice, stylus, touchscreens, microphones, audio capturing devices, or image/video capturing devices. An end user may utilize such input devices to interact with a user interface, for example a graphical user interface, for managing various functions performed by the computer 200.

The bus 206 may enable the processing unit 202 to read code and/or data to/from the mass storage device 212 or other computer-storage media. The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology. The computer-storage media may also represent secondary storage, whether implemented as hard drives or otherwise. Hard drive implementations may be characterized as solid state or may include rotating media storing magnetically-encoded information. The program modules 214, which include the imaging application 218, may include instructions that, when loaded into the processing unit 202 and executed, cause the computer 200 to provide functions associated with embodiments illustrated herein. The program modules 214 may also provide various tools or techniques by which the computer 200 may participate within the overall systems or operating environments using the components, flows, and data structures discussed throughout this description.

In general, the program modules 214 may, when loaded into the processing unit 202 and executed, transform the processing unit 202 and the overall computer 200 from a general-purpose computing system into a special-purpose computing system. The processing unit 202 may be constructed from any number of transistors or other discrete circuit elements, which may individually or collectively assume any number of states. More specifically, the processing unit 202 may operate as a finite-state machine, in response to executable instructions contained within the program modules 214. These computer-executable instructions may transform the processing unit 202 by specifying how the processing unit 202 transitions between states, thereby transforming the transistors or other discrete hardware elements constituting the processing unit 202.

Encoding the program modules 214 may also transform the physical structure of the computer-storage media. The specific transformation of physical structure may depend on various factors, in different implementations of this description. Examples of such factors may include but are not limited to the technology used to implement the computer-storage media, whether the computer storage media are characterized as primary or secondary storage, and the like. For example, if the computer-storage media are implemented as semiconductor-based memory, the program modules 214 may transform the physical state of the semiconductor memory, when the software is encoded therein. For example, the program modules 214 may transform the state of transistors, capacitors, or other discrete circuit elements constituting the semiconductor memory.

As another example, the computer-storage media may be implemented using magnetic or optical technology. In such implementations, the program modules 214 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 300) and software architectures that can be deployed in example embodiments.

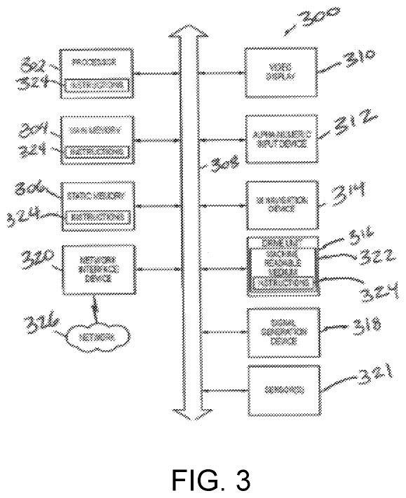

The machine 300 of FIG. 3 can operate as a standalone device or the machine 300 can be connected (e.g., networked) to other machines. In a networked deployment, the machine 300 can operate in the capacity of either a server or a client machine in server-client network environments. In an example, machine 300 can act as a peer machine in peer-to-peer (or other distributed) network environments. The machine 300 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 300. Further, while only a single machine 300 is illustrated, the term “machine” shall also be taken to include any collection of machines that individually or jointly execute a set (or multiple sets) of instructions to perform any one or more of the methodologies discussed herein.

Example machine (e.g., computer system) 300 can include a processor 302 (e.g., a central processing unit (CPU), a graphics processing unit (GPU) or both), a main memory 304 and a static memory 306, some or all of which can communicate with each other via a bus 308. The machine 300 can further include a display unit 310, an alphanumeric input device 312 (e.g., a keyboard), and a user interface (UI) navigation device 311 (e.g., a mouse). In an example, the display unit 810, input device 317 and UI navigation device 314 can be a touch screen display. The machine 300 can additionally include a storage device (e.g., drive unit) 316, a signal generation device 318 (e.g., a speaker), a network interface device 320, and one or more sensors 321, such as a global positioning system (GPS) sensor, compass, accelerometer, or other sensor. The storage device 316 can include a machine readable medium 322 on which is stored one or more sets of data structures or instructions 324 (e.g., software) embodying or utilized by any one or more of the methodologies or functions described herein. The instructions 324 can also reside, completely or at least partially, within the main memory 304, within static memory 306, or within the processor 302 during execution thereof by the machine 300. In an example, one or any combination of the processor 302, the main memory 304, the static memory 306, or the storage device 316 can constitute machine readable media. While the machine readable medium 322 is illustrated as a single medium, the term “machine readable medium” can include a single medium or multiple media (e.g., a centralized or distributed database, and/or associated caches and servers) that configured to store the one or more instructions 324. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media.

Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magnetooptical disks; and CD-ROM and DVD-ROM disks. The instructions 324 can further be transmitted or received over a communications network 326 using a transmission medium via the network interface device 320 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

To date, there has been limited work on wearable devices utilizing wavelengths across the visible spectrum for blood pressure measurement. Liu et. al [28] and Botrugno et. Al proposed multi-wavelength PPG algorithms for cuffless blood pressure monitoring, employing principal component analysis and neural networks, respectively. While these approaches have demonstrated performance comparable to, or even surpassing, single and dual-wavelength PPG sensors, they still face several limitations, including the need for individual calibration [28], operation within a relatively narrow spectral band [28] [29], reduced wearability [28], and limited interpretability of the resulting models [28] [29].

There is a growing body of work suggesting that additional contextual sensors that increase spectral resolution may improve optical biomarker detection, especially in the presence of noise [30]. In this paper, we introduce a multi-spectrum spectral framework using an off-the-shelf wearable for calibration-free BP monitoring. This framework employs interpretable features combined with machine learning techniques to estimate mean arterial blood pressure (MAP), systolic blood pressure (SBP), and diastolic blood pressure (DBP), achieving accuracy comparable to existing methods. This disclosure evaluates this framework in a n=9 feasibility study, spanning a range of BP values to demonstrate its effectiveness and generalizability.

Non-limiting contributions of this work include: (1) the development of a novel multi-spectrum spectroscopy framework that leverages wavelengths across the visible spectrum for calibration-free BP monitoring; (2) the implementation of classical machine learning techniques that utilize easily interpretable features to estimate MAP, SBP, and DBP, achieving accuracy comparable to traditional methods; and (3) a comprehensive evaluation of the proposed framework in a n=9 feasibility study to validate its effectiveness and generalizability.

An overview 100 of the system architecture used to collect multi-wavelength spectral data and convert these measurements to SBP, DBP, and MAP values relative to ground-truth is shown in FIG. 1.

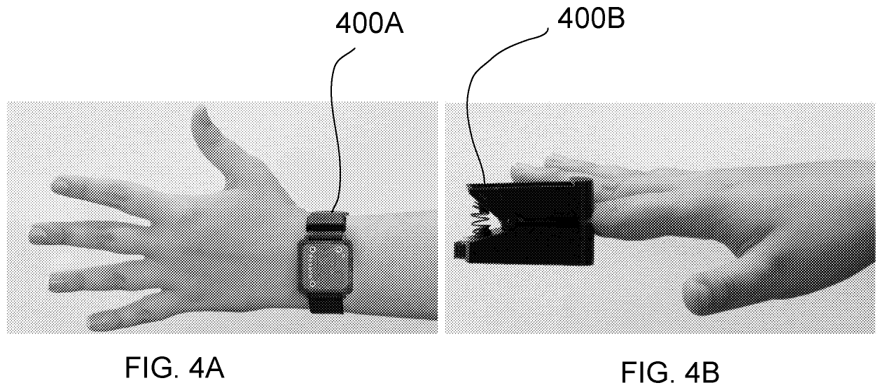

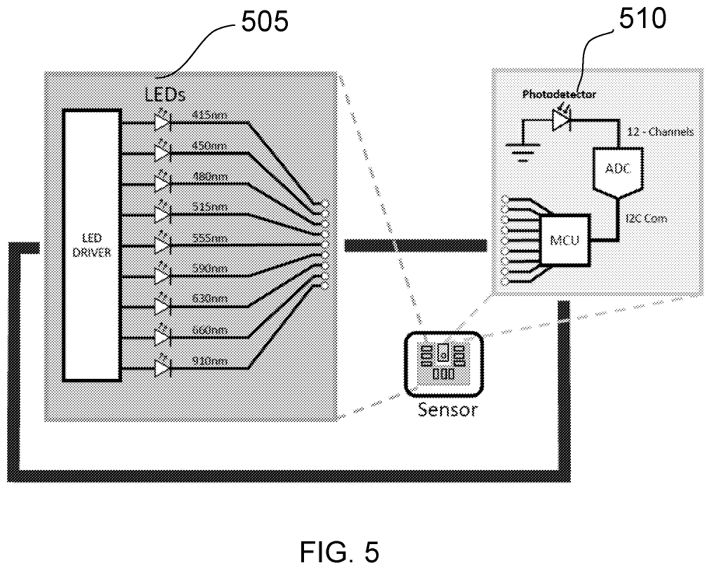

This disclosure utilized an example device called “Lumos” [31], an off-the-shelf wearable device, for multi-spectrum optical measurements. An example set up for the device used herein is shown in FIG. 5. Lumos utilizes LEDs 500 and photodetectors 510 in the form of photodiodes (PDs) ranging from 400 nm-1000 nm, allowing for a wide sensing spectral resolution that current commercial wearable devices do not provide. The device can be configured in several form-factors as shown in FIGS. 4A and 4B through the use of a detachable strap; in non-limiting examples, this disclosure utilized wrist form factor 400A and a fingertip cuff form factor 400B for this study. Regardless of configuration, spectral measurements are recorded in reflectance mode. Lumos functions by recording the PD responses for each LED that is turned on during its operation cyclically. Thus, for a sensor array of 9 PDs and 10 LEDs, there are 90 sensor readings every 1.7 seconds. These readings are arranged in an array and used as a feature vector for input to a machine learning algorithm. The machine learning algorithm may be implemented by a computer within a device that is worn or otherwise attached to the subject, or the machine learning algorithm can be implemented on a remote computer or server in wired or wireless communications with the device.

For ground-truth measurements, the BIOPAC NIBP100E was used [32]. The NIBP100E is a data acquisition system that uses arterial occlusion of the fingers along with occasional arm cuff calibration to provide continuous, beat-to-beat, blood pressure waveforms and downstream values (SBP, DBP, MAP). The system is FDA and CE-approved and is used commonly in academic settings, with an accuracy of +/−5 mm Hg. This disclosure conducted a proof-of-concept study with nine (9) subjects to evaluate feasibility of our framework. The BIOPAC NIBP100E was positioned on the participant's right arm, while an arm cuff was placed on the left arm for initial calibration. The Lumos devices (configured for finger and wrist placement) were worn on the left arm after calibration to avoid blood pressure distortions caused by upstream or downstream arterial occlusion from the NIBP finger cuffs. This setup allowed for accurate, distortion-free readings between arms, with continuous data collection from all devices.

To simulate varying blood pressure conditions, measurements were taken while participants were seated, standing, and lying down with their legs elevated. Each position was held for approximately 3 minutes, totaling 9 minutes per subject. During this time, the BIOPAC device continuously recorded SBP, DBP, and MAP values on a beat-to-beat basis. This study was approved by the Institutional Review Board of University of Virginia (IRB-HSR #240068).

Systolic, diastolic, and MAP values were time-aligned with the nearest respective Lumos measurements for direct comparison. The following features were then used for training on each row corresponding to a single Lumos measurement (90 columns) and output targets (SBP, DBP, MAP):
1) LED-PD values-representing sensor readings corresponding to individual LED and photodiode (PD) responses during a Lumos cycle.
2) LED-PD AC and DC extraction—5th-order high-pass and low-pass Butterworth [33] filters, with a cutoff frequency of 0.1 Hz, were applied to extract the alternating current (AC) and direct current (DC) components from each LED-PD signal.
3) Inter-beat interval—time between successive peaks for each LED-PD waveform.
4) Ratios of above features—to provide normalization or relational data, ratios of all the above features were taken.

These features were chosen based on the assumption that physiological factors—such as skin thickness, skin-tone, and vascular elasticity, which require re-calibration or advanced non-linear modeling in systems solely relying on traditional PPG, can be effectively captured using additional wavelengths that are utilized in Lumos. The simplicity of these features was intentional to prioritize interpretability.

To evaluate the performance of various machine learning models in predicting blood pressure, this disclosure used a combination of regression techniques. The tested models included tree-based algorithms such as random forest, gradient boosting, XGBoost, CatBoost, and LightGBM models, as well as linear models such as Ridge, Lasso, and basic linear regression. Variance thresholding was used to remove low-variance features, which were then scaled using RobustScaler, allowing the model to be resistant to outliers. Recursive feature elimination using a random forest model as the base estimator was used to reduce the feature set to the most relevant predictors prior to training. This disclosure utilized two models for each target variable per form-factor: a generalized model on the entire cohort and a subject-specific model that corrected for individual biases following training.

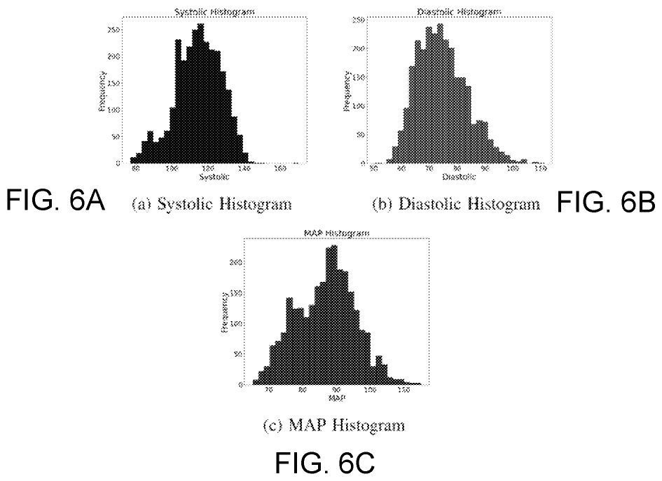

A summary of input statistics for the collected subject data can be found in FIGS. 6A, 6B, and 6C. The systolic values appear to follow a relatively normal distribution, with fewer data points observed in the hypertensive range. Diastolic values, conversely, are skewed towards lower blood pressure, while MAP displays a more symmetric, normal distribution.

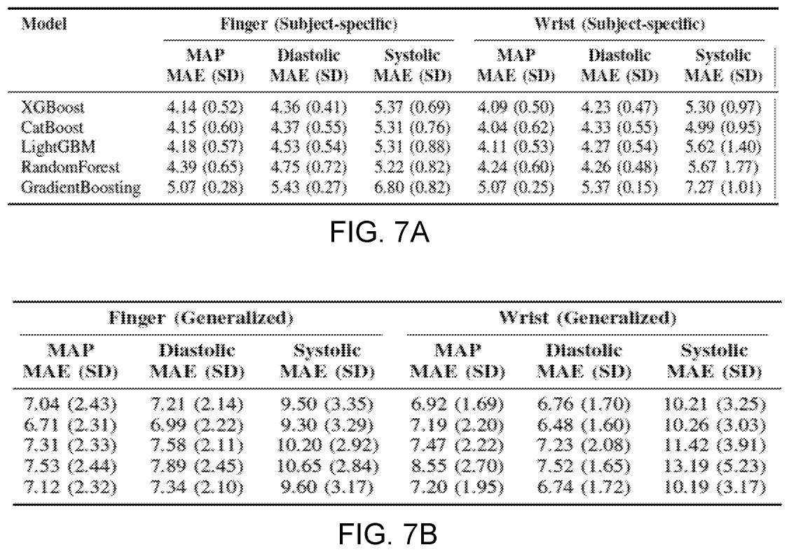

Given that the population is expected to be predominantly normotensive, these results align with expectations. This disclosure implemented leave-one-subject-out (LOSO) cross-validation to ensure the models had the ability to predict an entirely new subject's data after training. Mean absolute error (MAE) and its standard deviation (SD) were calculated for each model to quantify performance and model stability across various features and target variables. Table I shown in FIGS. 7A and 7B demonstrates the models'performance at predicting each blood pressure metric for the finger and wrist, using both generalized and subject-specific model. Performance is relatively strong across all models in both configurations, with MAP and diastolic MAEs out-performing systolic. The subject-specific models performed extremely well across form-factors and targets.

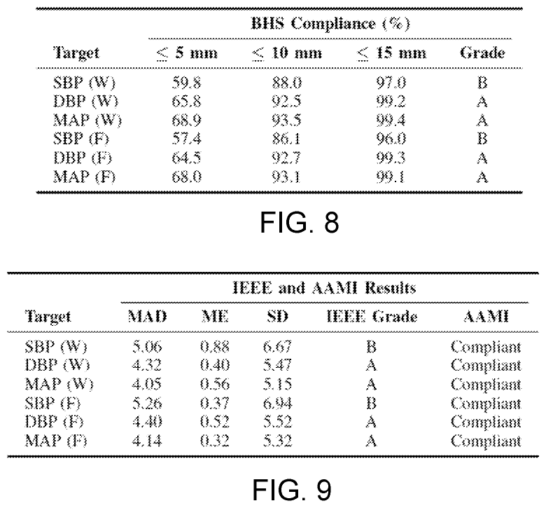

To contextualize these results, this disclosure presents them relative to current international standards. IEEE, the British Hypertension Society (BHS), and the Association for the Advancement of Medical Instrumentation (AAMI) each have standards regarding the performance of blood pressure estimation models; these standards are utilized by regulatory agencies during evaluation of these devices.
a) BHS classifies performance based on compliance percentage, or how much of a percentage of the predicted values fall within three thresholds: 5 mmHg, 10 mmHg, and 15 mmHg. The subject-specific wrist and finger systolic predictions score a B based on this classification, and an A for all other predictions. These results are summarized in Table II of FIG. 8.
b) IEEE and AAMI have similar standards, with different implementations. IEEE grades devices based on their mean absolute difference (MAD). Similar to the BHS classifications, embodiments herein score a B in subject-specific wrist and finger systolic predictions, and an A in all other predictions. Finally, AAMI determines compliance by checking the mean error (ME) as well as the standard deviation (SD). Across all predictions, embodiments of this disclosure are AAMI compliant. These results are summarized in Table III of FIG. 9.

The most important features to the models discussed herein were a combination of LED-PD DC extracted waveforms and their ratios in the 500-700 nm range, as well as LED-PD ratios across the visible spectrum and inter-beat internal (IBI) ratios in the 500-700 nm range. These features were consistent across formfactors, models, and targets. Below are examples of each:
Feature 1: LED633-PD590/LED670-PD415
Feature 2: LED415-PD680-DC/LED633-PD590-DC
Feature 3: LED633-PD590-IBI/LED670-PD415-IBI

The multi-wavelength optical sensing framework offers several advantages over traditional PPG-based systems for blood pressure estimation. By utilizing wavelengths across the visible spectrum, rather than relying on the traditional single or dual-wavelength approach, the system can allow for better differentiation between various tissue types and components, potentially increasing the accuracy of blood pressure estimation across diverse populations. A multi-wavelength approach may also be more robust against motion artifacts and environmental interference, since all channels may not be affected by noise. Furthermore, the framework is extremely versatile: the device can be fit into multiple form-factors, not just the finger and wrist, and can be equipped with sensors from across the visible and infrared spectrum to tailor sensing using wavelengths that are specific to blood pressure detection.

The subject models have uncovered features that suggest a deeper understanding of tissue properties beyond those measured by traditional PPG, potentially improving blood pressure detection.

Given the particular relevance of feature ratios, its possible that DC, LED-PD, and IBI ratios capture complementary aspects of light interaction that are not solely correlated with the pulse wave. Notably, while IBI is traditionally associated with heart rate, in this context, it may also capture other AC components related to respiration and skin dynamics, thus providing a normalization to traditional PPG features and improving overall blood pressure detection. Further studies will be conducted to explore these relationships in greater detail.

While the current models available for this disclosure benefit from subject-specific training, it is expected that general-purpose models will perform similarly with sufficient data. While subject-specific models may resemble traditional calibration, they likely learn spectral patterns specific to the individual, thereby eliminating the need for periodic recalibration as required in methods like pulse transit time and allowing for a one-time calibration. Overall, as a training dataset grows, it is expected that generalized models will achieve performance comparable to subject-specific models. This disclosure also observed significant oversaturation in certain photodiodes during the trials, particularly the 820 nm PD, which might be relevant for blood pressure detection; one aim is to correct this in future studies and expect better performance as a result.

While this study serves as a promising proof of concept for our multi-wavelength optical sensing framework, significant further validation is will be forthcoming. Suggested next steps include expanding the study to a much larger and more diverse cohort, encompassing a wider range of blood pressure values, with particular emphasis on hypertensive subjects, as well as various demographic factors including age, gender, and fitness levels. The devices of this disclosure will also investigate edge cases by exploring blood pressure changes in scenarios such as drug administration effects, stress-induced fluctuations, and exercise-related variations. Given that blood pressure typically decreases during sleep [34], an upcoming trial will conduct overnight studies to evaluate the system's performance in detecting and accurately measuring these nocturnal dips. To ensure long-term stability, this disclosure will also assess the algorithm's accuracy and reliability over extended periods to confirm consistent performance without the need for recalibration. Lastly, additional goals for this research include conducting rigorous comparisons against invasive arterial line measurements and other FDA-approved non-invasive devices to validate our system's accuracy.

This paper introduces a novel multi-wavelength optical sensing framework for calibration-free, wearable blood pressure monitoring. By leveraging multiple wavelengths across the visible spectrum, combined with machine learning and signal processing techniques, the research herein has demonstrated the ability of the system to provide accurate estimates of SBP, DBP, and MAP. The results of the proof-of-concept study (n=9) indicate that the proposed framework achieves accuracy comparable to current cuff-based methods while maintaining interpretability and robustness across a range of blood pressures. Future work will focus on expanding the cohort size to include a more diverse demographic and exploring the framework's performance in various scenarios, such as during physical activity or in response to pharmacological interventions. Additionally, further validation against invasive and non-invasive gold standard devices will be necessary to confirm the system's clinical applicability.

In one implementation, a computer implemented method of measuring blood pressure of a subject includes placing a device on a surface of the subject in a position that is proximate to blood vessels identified for blood pressure measurements. The device directs a plurality of wavelengths of light from the device toward the identified blood vessels. The method continues by generating a plurality of respective photoplethysmography (PPG) waveforms and corresponding PPG data with the device in response to each of the wavelengths of light, and for each of the wavelengths of light, transmitting wavelength data and the corresponding PPG data from the device to a computer comprising a processor in communication with computer memory storing the wavelength data and the PPG data in a feature vector. The method further includes the processor conducting steps of extracting feature values from the feature vector; calculating feature ratios of selected pairs of feature values that each comprise a corresponding PPG data value for a respective wavelength data value; and using the feature ratios as inputs in a machine learning model to predict systolic blood pressure (SBP), diastolic blood pressure (DBP), or mean arterial blood pressure (MAP) with the feature ratios.

The computer implemented method further includes directing the plurality of wavelengths of light with respective light emitting diodes (LEDs) specified at a respective wavelength; generating the PPG waveforms with a plurality of photodetectors positioned within the light transmission path of the respective LEDs; extracting the feature values from individual photodetector responses paired with individual LED wavelength data, AC components of the PPG waveforms, DC components of the PPG waveforms, or inter-beat interval values.

The computer implemented method further includes storing the wavelength data and the corresponding PPG data in a feature vector comprising rows of the wavelength data and columns of the corresponding PPG data; and selecting the pairs of feature values for the feature ratios from the feature vector.

The computer implemented method further includes directing the plurality of wavelengths of light with respective light emitting diodes (LEDs) specified at a respective wavelength.

The computer implemented method further includes generating the PPG waveforms with a plurality of photodetectors positioned within the light transmission path of the respective LEDs.

The feature value comprises at least one feature value selected from individual photodiode responses paired with individual LED wavelength data, extracted AC components of the PPG waveforms, extracted DC components of the PPG waveforms, and inter-beat interval values.

The computer implemented method further includes operating the photodetectors in transmission mode (e.g. a finger clip) or reflectance mode (e.g. a wrist watch), and operating the LEDs in the visible spectrum.

The computer implemented method further includes operating the LEDs in the 500 nm to 700 nm range, inclusive.

The computer implemented method further includes placing a wearable device on the skin of the subject.

The computer implemented method further includes positioning the wearable device on an arm, a wrist, or a finger of the subject.

The computer implemented method further includes gathering ground truth data for comparing systolic blood pressure (SBP), diastolic blood pressure (DBP), or mean arterial blood pressure (MAP) with corresponding values predicted by the machine learning model with the feature ratios.

The computer implemented method further includes selecting a machine learning model from tree-based algorithms or linear models and training the machine learning model with a plurality of feature vectors comprising the ground truth data and.

The computer implemented method further includes reducing the feature vector to most relevant predictors prior to training the machine learning model with corresponding feature vectors comprising tested wavelength data and tested PPG data from prior uses of the device.

The computer implemented method further includes using the machine learning model to learn spectral patterns for the subject using the device.

The computer implemented method may utilize the LEDs and the photodetectors that operate in the infrared spectrum in an optional embodiment.

In another implementation, an apparatus for measuring blood pressure of a subject includes a form factor configured to place the apparatus on a surface of the subject in a position that is proximate to blood vessels identified for blood pressure measurements. A plurality of LEDs direct a plurality of wavelengths of light from the apparatus toward the identified blood vessels. A plurality of photodetectors generating a plurality of respective photoplethysmography (PPG) waveforms and corresponding PPG data in response to each of the wavelengths of light, and the photodetectors are configured to transmit, for each of the wavelengths of light, wavelength data and the corresponding PPG data from the apparatus to a computer comprising a processor in communication with computer memory storing the wavelength data and the PPG data in a feature vector. Software implemented by the computer perform steps including extracting feature values from the feature vector; calculating feature ratios of selected pairs of feature values that each comprise a corresponding PPG data value for a respective wavelength data value; and using the feature ratios as inputs in a machine learning model to predict systolic blood pressure (SBP), diastolic blood pressure (DBP), or mean arterial blood pressure (MAP) with the feature ratios.

The steps implemented by the device include extracting the feature values from individual photodetector responses paired with individual LED wavelength data, AC components of the PPG waveforms, DC components of the PPG waveforms, or inter-beat interval values. Additional embodiments of this disclosure employ not only a plurality of LED wavelengths, but a plurality of photodiode readings across the visible spectrum. It has been determined that mismatched LED-PD data collection improves data collection under different environmental/physiological contexts. The method of this disclosure also incorporates adding not just feature ratios, but temporal and spectral features as well. The computer implemented method includes incorporation of data regarding inter-beat intervals between LED and photodiode readings. Additionally, the computer implemented method includes the use of spectral gradient slopes, whereby a hyperplane is overlayed across the visible spectrum and differential relationships between LED-photodiode pairs are taken.

The form factor of the apparatus is configured for wearing on the subject's wrist, arm or finger.

The computer implemented method may be configured a computer program product for implementation by other devices.

### References

[1] World Health Organization, “World health statistics: a snapshot of global health,” Organizacion Mundial de la Salud, Tech. Rep., 2012.
[2] Centers for Disease Control and Prevention, “High blood pressure facts and statistics”; U.S Department of Health and Human Services. [Online]. Available: https://www.cdc.gov/high-blood-pressure/data-research/factsstats/index.htm.
[3] S. M. Hamrahian et al., “A critical review of medication adherence in hypertension: Barriers and facilitators clinicians should consider,” Patient preference and adherence, vol. 16, pp. 2749-2757, 2022.
[4] J. E. Sharman et al., “Automated Oscillo-etric blood pressure measuring devices: how they work and what they measure,” Journal of Human Hypertension, vol. 37, no. 2, pp. 93-100, 2023.
[5] D. M. Bard, J. I. Joseph, and N. van Helmond, “Cuff-less methods for blood pressure telemonitoring,” Frontiers in Cardiovascular Medicine, vol. 6, p. 40, 2019.
[6] G. S. Stergiou et al., “Accuracy of automated cuff blood pressure monitors in special populations: International organization for standardization (iso) task group report and call for research,” Journal of Hypertension, vol. 41, no. 5, pp. 811-818, 2023.
[7] J. Ishigami et al., “Effects of cuff size on the accuracy of blood pressure readings: The cuff(sz) randomized crossover trial,” JAMA Internal Medicine, vol. 183, no. 10, pp. 1061-1068, 2023.
[8] H. Badeli and F. Assadi, “Strategies to reduce pitfalls in measuring blood pressure,” International Journal of Preventive Medicine, vol. 5, no. Suppl 1, pp. S17-S20, March 2014.
[9] K. Eguchi et al., “What is the optimal interval between successive home blood pressure readings using an automated Oscillo-Metric device?” Journal of Hypertension, vol. 27, no. 6, pp. 1172-1177, 2009.
[10] M. Y. Cheung, A. Sabharwal, G. L. Cote, and A. Veeraraghavan, “Wearable blood pressure monitoring devices: Understanding heterogeneity in design and evaluation,” IEEE Transactions on Biomedical Engineering, 2024.
[11] P. K. Man et al., “Blood pressure measurement: From cuff-based to contactless monitoring,” Healthcare (Basel, Switzerland), vol. 10, no. 10, p. 2113, Oct. 2022. [Online]. Available: https://doi.org/10.3390/healthcare10102113.
[12] L. A. Geddes, M. H. Voelz, C. F. Babbs, J. D. Bourland, and W. A. Tacker, “Pulse transit time as an indicator of arterial blood pressure,” Psychophysiology, vol. 18, no. 1, pp. 71-74, Jan. 1981. [Online]. Available: https://doi.org/10.1111/j.1469-8986.1981.tb01545.x.
[13] Y. Zhang, Y. Zheng, Z. Ma, H. Wang, and J. Luo, “Radial pulse transit time is an index of arterial stiffness,” Hypertension Research, vol. 34, pp. 884-887, 2011. [Online]. Available: https://doi.org/10.1038/hr.2011.41.
[14] R. C. Block, P. A. Steptoe, S. L. Evans, G. G. Akingba, R. F. Glover, D. A. Malinow, and B. R. Hemminger, “Conventional pulse transit times as markers of blood pressure changes in humans,” Scientific Reports, vol. 10, no. 1, p. 16373, Oct. 2020. [Online]. Available: https://doi.org/10.1038/s41598-020-73143-8.
[15] R. Mukkamala, M. Yavarimanesh, K. Natarajan, J. -O. Hahn, K. G. Kyriakoulis, A. P. Volio, and G. S. Stergiou, “Evaluation of the accuracy of cuffless blood pressure measurement devices: challenges and proposals,” Hypertension, vol. 78, no. 5, pp. 1161-1167, 2021.
[16] A. Avolio et al., “Challenges presented by cuffless measurement of blood pressure if adopted for diagnosis and treatment of hypertension, “Pulse (Basel, Switzerland), vol. 10, no. 1-4, pp. 34-45, Apr. 2022. [Online]. Available: https://doi.org/10.1159/000522660.
[17] J. -R. Hu et al., “Validating cuffless continuous blood pressure monitoring devices,” Cardiovascular Digital Health Journal, vol. 4, no. 1, pp. 9-20, Jan. 2023. [Online]. Available: https://doi.org/10.1016/j.cvdhj.2023.01.001.
[18] X. Zhou et al., “Validation of new and existing decision rules for the estimation of beat-to-beat pulse transit time,” BioMed Research International, vol. 2015, p. 306934, 2015. [Online]. Available: https://doi.org/10.1155/2015/306934.
[19] Y. Ma et al., “Relation between blood pressure and pulse wave velocity for human arteries,” Proceedings of the National Academy of Sciences of the United States of America, vol. 115, no. 44, pp. 11 144-11 149, 2018. [Online]. Available: https://doi.org/10.1073/pnas.1814392115.
[20] S. G. Khalid, J. Zhang, F. Chen, and D. Zheng, “Blood pressure estimation using photoplethysmography only: Comparison between different machine learning approaches,” Journal of Healthcare Engineering, vol. 2018, p. 1548647, 2018. [Online]. Available: https://doi.org/10.1155/2018/1548647.
[21] M. Kachuee, M. M. Kiani, H. Mohammadzade, and M. Shabany, “Cuffless high-accuracy calibration-free blood pressure estimation using pulse transit time,” in 2015 IEEE International Symposium on Circuits and Systems (ISCAS), 2015, pp. 1006-1009.
[22] H. Samimi and H. R. Dajani, “A ppg-based calibration-free cuffless blood pressure estimation method using cardiovascular dynamics,” Sensors (Basel, Switzerland), vol. 23, no. 8, p. 4145, Apr. 2023. [Online]. Available: https://doi.org/10.3390/s23084145.
[23] Y. Seo et al., “Blood pressure estimation and its recalibration assessment using wrist cuff blood pressure monitor,” Biomedical Engineering Letters, vol. 13, no. 2, pp. 221-233, Mar. 2023. [Online]. Available: https://doi.org/10.1007/s13534-023-00271-1.
[24] Aktiia, “Aktiia wins ce mark for calibration-free optical blood pressure measuring tech,” 2024. [Online]. Available: https://www.medicaldevicenetwork.com/news/aktiia-ce-mark-calibration-free-bp-tech/.
[25] C. Health, “Corsano health's calibration-free blood pressure monitoring with ppg technology,” 2024. [Online]. Available: https://corsano.com/corsano-resources/nibp/.
[26] Valencell, “Valencell expands worlf's first calibration-free, cuffless blood pressure monitoring system to the finger and wrist,” 2021. [Online]. Available: https://valencell.com/news/valencell-expandsworlds-first-calibration-free-cuffless-blood-pressure-monitoring-systemto-the-finger-and-wrist.
[27] H. S. Saad, J. F. W. Zaki, and M. M. Abdelsalam, “Employing of machine learning and wearable devices in healthcare system: Tasks and challenges,” Neural Computing and Applications, 2024. [Online]. Available: https://doi.org/10.1007/s00521-024-10197-z
[28] J. Liu, S. Qiu, N. Luo, S. -K. Lau, H. Yu, T. Kwok, Y. -T. Zhang, and N. Zhao, “Pca-based multi-wavelength photoplethysmography algorithm for cuffless blood pressure measurement on elderly subjects,” IEEE Journal of Biomedical and Health Informatics, vol. 25, no. 3, pp. 663-673, 2021.
[29] C. Botrugno, K. Dheman, P. Bonazzi, F. Dell'Olio, and M. Magno, “AI-based multi-wavelength ppg device for blood pressure monitoring” in 2024 IEEE International Symposium on Medical Measurements and Applications (MeMeA), 2024, pp. 1-6.
[30] D. Ray, T. Collins, S. I. Woolley, and P. V. S. Ponnapalli, “A review of wearable multi-wavelength photoplethysmography,” IEEE Reviews in Biomedical Engineering, vol. 16, pp. 136-151, 2023.
[31] A. Watson, C. Kendell, A. Lingamoorthy, I. Lee, and J. Weimer, “Lumos: An open-source device for wearable spectroscopy research,” Proc. ACM Interact. Mob. Wearable ubiquitous Technol., vol. 6, no. 4, Jan. 2023. [Online]. Available: https://doi.org/10.1145/3569502.
[32] BIOPAC Systems, Inc., “Noninvasive blood pressure amplifier,” 2024. [Online]. Available: https://www.biopac.com/product/noninvasiveblood-pressure-amplifier.
[33] S. Butterworth, “On the theory of filter amplifiers,” xperimental wireless and the wireless engineer, vol. 7, pp. 536-541, 1930.
[34] M. H. Smolensky and E. Haus, “Circadian rhythms and clinical medicine with applications to hypertension,” American Journal of Hypertension, vol. 14, pp. 280S-290S, 2001.

1. A computer implemented method of measuring blood pressure of a subject, comprising:
placing a device on a surface of the subject in a position that is proximate to blood vessels identified for blood pressure measurements;
directing a plurality of wavelengths of light from the device toward the identified blood vessels;
generating a plurality of respective photoplethysmography (PPG) waveforms and corresponding PPG data with the device in response to each of the wavelengths of light;
for each of the wavelengths of light, transmitting wavelength data and the corresponding PPG data from the device to a computer comprising a processor in communication with computer memory storing the wavelength data and the PPG data in a feature vector;
extracting feature values from the feature vector;
calculating feature ratios of selected pairs of feature values that each comprise a corresponding PPG data value for a respective wavelength data value; and
using the feature ratios as inputs in a machine learning model to predict systolic blood pressure (SBP), diastolic blood pressure (DBP), or mean arterial blood pressure (MAP) with the feature ratios.

2. The computer implemented method of claim 1, further comprising:
directing the plurality of wavelengths of light with respective light emitting diodes (LEDs) specified at a respective wavelength;
generating the PPG waveforms with a plurality of photodetectors positioned within the light transmission path of the respective LEDs;
extracting the feature values from individual photodetector responses paired with individual LED wavelength data, AC components of the PPG waveforms, DC components of the PPG waveforms, or inter-beat interval values.

3. The computer implemented method of claim 1, further comprising storing the wavelength data and the corresponding PPG data in a feature vector comprising rows of the wavelength data and columns of the corresponding PPG data; and
selecting the pairs of feature values for the feature ratios from the feature vector.

4. The computer implemented method of claim 1, further comprising directing the plurality of wavelengths of light with respective light emitting diodes (LEDs) specified at a respective wavelength.

5. The computer implemented method of claim 4 further comprising generating the PPG waveforms with a plurality of photodetectors positioned within the light transmission path of the respective LEDs.

6. The computer implemented method of claim 5, wherein the feature value comprises at least one feature value selected from individual photodiode responses paired with individual LED wavelength data, extracted AC components of the PPG waveforms, extracted DC components of the PPG waveforms, and inter-beat interval values.

7. The computer implemented method of claim 6, further comprising operating the photodetectors in transmission mode or reflectance mode, and operating the LEDs in the visible spectrum.

8. The computer implemented method of claim 7, further comprising operating the LEDs in the 500 nm to 700 nm range, inclusive.

9. The computer implemented method of claim 7, further comprising gathering ground truth data for comparing systolic blood pressure (SBP), diastolic blood pressure (DBP), or mean arterial blood pressure (MAP) with corresponding values predicted by the machine learning model with the feature ratios.

10. The computer implemented method of claim 1, further comprising placing a wearable device on the skin of the subject.

11. The computer implemented method of claim 10, further comprising positioning the wearable device on an arm, a wrist, or a finger of the subject.

12. The computer implemented method of claim 1, further comprising selecting a machine learning model from tree-based algorithms or linear models and training the machine learning model with a plurality of feature vectors comprising the ground truth data and.

13. The computer implemented method of claim 12, further comprising reducing the feature vector to most relevant predictors prior to training the machine learning model with corresponding feature vectors comprising tested wavelength data and tested PPG data from prior uses of the device.

14. The computer implemented method of claim 12, further comprising using the machine learning model to learn spectral patterns for the subject using the device.

15. The computer implemented method of claim 1, wherein the LEDs and the photodetectors operate in the infrared spectrum.

16. An apparatus for measuring blood pressure of a subject, comprising:
a form factor configured to place the apparatus on a surface of the subject in a position that is proximate to blood vessels identified for blood pressure measurements;
a plurality of LEDs directing a plurality of wavelengths of light from the apparatus toward the identified blood vessels;
a plurality of photodetectors generating a plurality of respective photoplethysmography (PPG) waveforms and corresponding PPG data in response to each of the wavelengths of light, and the photodetectors being configured to transmit, for each of the wavelengths of light, wavelength data and the corresponding PPG data from the apparatus to a computer comprising a processor in communication with computer memory storing the wavelength data and the PPG data in a feature vector;
software implemented by the computer to perform steps comprising:
extracting feature values from the feature vector;
calculating feature ratios of selected pairs of feature values that each comprise a corresponding PPG data value for a respective wavelength data value; and
using the feature ratios as inputs in a machine learning model to predict systolic blood pressure (SBP), diastolic blood pressure (DBP), or mean arterial blood pressure (MAP) with the feature ratios.

17. The apparatus of claim 16, wherein the steps further comprise the computer extracting the feature values from individual photodetector responses paired with individual LED wavelength data, AC components of the PPG waveforms, DC components of the PPG waveforms, or inter-beat interval values.

18. The apparatus of claim 16, wherein the form factor of the apparatus is configured for wearing on the subject's wrist, arm or finger.

19. The apparatus of claim 16, wherein the feature vector includes data regarding inter-beat intervals between LED and photodiode readings.

20. The apparatus of claim 16, further comprising utilizing the computer calculate and save spectral gradient slopes by modeling a hyperplane overlayed across the visible spectrum and calculating differential relationships between LED-photodiode pairs for use in the feature vector.
